(19) United States
(12) Patent Application Publication
(10) Pub. No.: US 2025/0268529 A1
(43) Pub. Date: Aug. 28, 2025

(54) ORTHOPEDIC IMPLANT AND RELATED ELECTRODE ASSEMBLY

(71) Applicant: GLOBUS MEDICAL, INC., AUDUBON, PA (US)

(72) Inventors: Paden Troxell, Conshohocken, PA (US); David C. Paul, Phoenixville, PA (US)

(21) Appl. No.: 18/589,785

(22) Filed: Feb. 28, 2024

(51) Int. Cl. A61B 5/00

(52) U.S. Cl. CPC A61B 5/4851; A61B 5/0022; A61B 5/0031; A61B 5/686; A61B 2560/0406; A61B 2560/045; A61B 2560/0468; A61B 2562/0215; A61B 2562/043; A61B 2562/164

(57) ABSTRACT

Various implementations include orthopedic implants and related electrode assemblies. In a particular implementation, an electrode assembly includes: a first housing containing at least one electrode; and a control module with a first connector for mating with a complementary connector on the first housing, the control module including a controller programmed to initiate transmission of at least one of a measurement pattern or a stimulation pattern to a patient with the at least one electrode, wherein the first housing and the control module include a biocompatible material, and when connected, are sized to complement an orthopedic implant.

### TECHNICAL FIELD

This disclosure generally relates to the field of surgery and surgical implants. More particularly, the disclosure relates to implants, assemblies, and related systems enabling treatment and/or monitoring of post-operative patient progress.

### BACKGROUND

A promising frontier in orthopedics is the conception and commercialization of smart implants. In the context of orthopedics, the term “smart implant” describes a traditional orthopedic implant with enhanced functionality enabled by integrated sensing, processing, communication, power, actuation, and/or treatment functions. Conventional sensing and stimulation functions for smart implants involve the placement of one or more conductive elements (i.e. electrodes) at the site of interest. However, conventional procedures for placing smart components such as sensors, actuators, processing equipment, etc., can be cumbersome or unnecessarily invasive.

### SUMMARY

The needs above, as well as others, are addressed by embodiments of apparatuses for providing feedback on implants, as well as systems for providing implant feedback, and related methods described in this disclosure. All examples and features mentioned below can be combined in any technically possible way.

Various implementations include orthopedic implants and related electrode assemblies adapted for use with such orthopedic implants. In a particular implementation, an electrode assembly includes: a first housing containing at least one electrode; and a control module with a first connector for mating with a complementary connector on the first housing, the control module including a controller programmed to initiate transmission of at least one of a measurement pattern or a stimulation pattern to a patient with the at least one electrode, where the first housing and the control module include a biocompatible material, and when connected, are sized to complement an orthopedic implant.

In particular aspects, an orthopedic implant includes: a body for securing in a patient, the body including a mating feature; and an electrode assembly coupled with the body at the mating feature, the electrode assembly including: a first housing containing at least one electrode; and a control module connected with the first housing, the control module including a controller programmed to initiate transmission of at least one of a measurement pattern or a stimulation pattern to a patient with the at least one electrode, where the electrode assembly complements the mating feature on the body.

Implementations may include one of the following features, or any combination thereof.

In some examples, the biocompatible material includes an electrically insulative biocompatible material. In particular examples, the biocompatible material includes a rigid material such as PEEK (polyether ether ketone), HXLPE (highly cross-linked polyethylene) and/or a flexible material such as silicone, TPU (thermoplastic polyurethane), or PTFE (polytetrafluoroethylene). In additional cases, the biocompatible material includes an implantable metal such as stainless steel or titanium, that is electrically isolated from the electrodes.

In certain examples, the controller is programmed to initiate transmission of both the measurement pattern and the stimulation pattern to the patient, and the at least one electrode is configured for use in both the measurement pattern and the stimulation pattern.

In particular cases, the first housing contains a plurality of electrodes.

In some aspects, the electrode assembly further includes a second housing containing at least one additional electrode, where the control module includes a second connector for mating with a complementary connector on the second housing.

In certain implementations, when the first housing and the control module are connected, the electrode assembly is pliable to complement at least one contour in the orthopedic implant.

In some cases, the electrode assembly is sized to sit within a groove or slot in the orthopedic implant.

In particular aspects, the first housing includes a flexible insulative section adjacent the at least one electrode.

In some implementations, the first housing includes a plurality of electrodes interposed between adjacent insulative sections, where each of the plurality of electrodes is selectable for at least one of: measurement based on the measurement pattern or stimulation based on the stimulation pattern.

In certain cases, the plurality of electrodes includes at least three electrodes, where according to the stimulation pattern: a first electrode is selected as an anode and a second electrode is selected as a cathode. In certain cases, additional electrodes are selected as one of an anode or a cathode according to the stimulation pattern. In accordance with the measurement pattern: a first electrode is selected as a working electrode, a second electrode is selected as a counter electrode, and a third electrode is selected as a reference electrode. In some examples, the first, second, and third electrode form an electrochemical cell in the measurement pattern.

In particular cases, the controller is configured to change selection of one or more of the plurality of electrodes according to the stimulation pattern or the measurement pattern.

In certain aspects, the first housing includes a flexible tubular body and the at least one electrode is integrated in a wall of the flexible tubular body such that a primary axis of each electrode is parallel with a primary axis of the flexible tubular body. In various examples, the primary axis is the long axis.

In some cases, the first housing includes a flexible tubular body with an internal channel, and the at least one electrode is integrated in a wall of the flexible tubular body such that a primary axis of each electrode is off-axis relative to a primary axis of the flexible tubular body. In some cases, the internal channel contains wiring, e.g., internal wiring. In other cases, wiring is integrated in or otherwise connected with the walls. In certain cases, multiple electrodes are integrated in a wall of the flexible tubular body and are either evenly spaced relative to one another or are unevenly spaced relative to one another. In various examples, the off-axis electrodes are approximately parallel to the primary axis of the flexible tubular body.

In certain cases, the control module includes one or more application specific integrated circuits (ASICs).

In particular cases, the control module includes a hermetically sealed housing constructed from an implantable metal or a biocompatible polymer. In some cases, the implantable metal includes titanium. In certain examples, the biocompatible polymer includes PEEK. In various implementations, the housing includes: the controller, memory, a multiplexer, a power system, and a communication system, wherein the power system enables wireless power transfer to the control module and the communication system enables wireless communication with an external control device. In some cases, wireless communication includes communication with one or more protocols, for example, Bluetooth (BT), BT Low Energy (BLE), radio frequency (RF), etc. In particular cases, the control module is connected with a local control and/or display device such as a smart phone or smart device (e.g., tablet, computing device, etc.) that has network connectivity. In some examples, the local control and/or display device can be configured to communicate over a network to transmit treatment instructions to the control module and/or receive sensor data for remote transmission.

In certain aspects, the first housing and the control module have a substantially arcuate outer dimension or a substantially angled outer dimension. In some cases, the substantially angled outer dimension includes a tubular outer dimension, a rectangular outer dimension, or an oblong outer dimension. In particular cases, each electrode can have a shape corresponding with an outer dimension of the control module. In particular cases, the combined assembly has a linear shape or a toroidal shape. In certain cases, the shape of electrodes and/or the control module can be tailored to fit in the orthopedic implant, e.g., within a slot or opening in the implant. For example, a slot with angled walls or arcuate walls can be fit with an assembly that has angled outer walls or arcuate outer walls.

In some examples, the orthopedic implant includes a knee implant, a tibial insert, a hip implant, or a surgical nail.

In particular cases, the first housing and the control module are integrally assembled, or configured for assembly during a surgical procedure on the patient.

In other examples, the electrode assembly and control module are provided separately and assembled during the surgical procedure, e.g., in the surgical theater or intraoperatively. In certain examples, each electrode assembly has an electrical connector component with one or more conductive surfaces (e.g., pads) that are individually electrically coupled to each electrode. In certain cases, the electrode assembly is securely attached to mating contacts on the control module housing (or, canister), via mechanical means such as with set screws or pins.

In certain aspects of the orthopedic implant, the first housing includes a plurality of electrodes interposed between adjacent insulative sections, where each of the plurality of electrodes is selectable for at least one of: measurement based on the measurement pattern or stimulation based on the stimulation pattern.

In further aspects of the orthopedic implant, the controller is programmed to initiate transmission of both the measurement pattern and the stimulation pattern to a patient, and the at least one electrode is configured for use in both the measurement pattern and the stimulation pattern.

In additional aspects of the orthopedic implant, the mating feature includes a groove or a slot in the body, and the electrode assembly is retained in the groove or slot in the body.

Two or more features described in this disclosure, including those described in this summary section, may be combined to form implementations not specifically described herein.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

The details of one or more implementations are set forth in the accompanying drawings and the description below. Other features, objects and benefits will be apparent from the description and drawings, and from the claims.

### BRIEF DESCRIPTION OF THE DRAWINGS

FIG. 1 shows a schematic system view of an implant according to various implementations.

FIG. 2 shows an example electrode assembly according to various implementations.

FIG. 3 shows an example electrode assembly according to various additional implementations.

FIGS. 4A-4C show example electrode assemblies according to various further implementations.

FIG. 5 shows a perspective view of circular or oblong electrode assembly according to various implementations.

FIG. 6 shows a perspective view of an electrode assembly including two distinct electrode housings according to various implementations.

FIG. 7 is a system diagram of a control module according to various implementations.

FIG. 8 illustrates a variation on the control module of FIG. 7 according to various implementations.

FIG. 9 shows a tubular form of an electrode housing according to various implementations.

FIG. 10 shows a rectangular form of an electrode housing according to various additional implementations.

FIGS. 11A-11C and 12A-12C illustrate control functions in electrode measurement and/or stimulation according to various implementations.

FIGS. 13A-13D show applications of electrode assemblies in an implant according to various implementations.

FIG. 14 is a perspective view of an electrode assembly for an implant according to various additional implementations.

FIGS. 15A-15B show views of an implant with a slot or recess for receiving an electrode assembly according to various implementations.

FIG. 16 shows an electrode assembly in an implant according to one of various implementations.

FIG. 17 shows an electrode assembly in an implant according to various additional implementations.

FIGS. 18A-18B show an electrode assembly in two distinct implants according to various additional implementations.

FIGS. 19A-19B illustrates flexible tubular electrode assemblies in two distinct implants according to various additional implementations.

FIG. 20 shows a type of flexible tubular electrode assembly according to various implementations.

FIG. 21 shows another type of flexible tubular electrode assembly according to various implementations.

FIG. 22 shows a system for enabling control of an implant according to various implementations.

It is noted that the drawings of the various implementations are not necessarily to scale. The drawings are intended to depict only typical aspects of the disclosure, and therefore should not be considered as limiting the scope of the implementations. In the drawings, like numbering represents like elements between the drawings.

### DETAILED DESCRIPTION

Various example embodiments of assemblies and implants with integrated electrodes and control modules for controlling transmission of a measurement pattern and/or a stimulation pattern to a patient are described herein. Various particular implementations include orthopedic implants such as a knee implant, a tibial insert, a hip implant, or a surgical nail with an integrated electrode assembly.

In the interest of clarity, not all features of an actual implementation are necessarily described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The apparatuses and related systems and methods described herein boast a variety of inventive features and components that warrant patent protection, both individually and in combination.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

As noted herein, the various disclosed implementations can reduce complexity and/or invasiveness of conventional surgical procedures, for example, by attaching the electrodes on or within the surface of the implant form. Further, relative to conventional implants, the disclosed implementations efficiently allocate electrodes and control modules, and also provide one or more separate components that can be assembled to traditional implant components within the surgical theater. Various implementations include assemblies, implants, systems and methods of coupling electrodes to smart orthopedic implant systems.

This disclosure provides, at least in part, an electrode assembly sized to complement an orthopedic implant and a related orthopedic implant. Various implementations of the electrode assembly allow a medical professional such as a surgeon to insert or otherwise affix the electrode assembly to an orthopedic implant, e.g., in a surgical theater or intraoperatively. Various implementations include an implant with an electrode assembly that can be used to initiate at least one of a measurement pattern or a stimulation pattern to a patient. The various disclosed implementations can improve patient outcomes when compared with conventional implants. The disclosed implementations can provide post-operative feedback on implant procedures and healing, as well as a means to stimulate healing post-operatively. The disclosed implementations can enhance both current procedural outcomes as well as future surgical outcomes.

Commonly labeled components in the FIGURES are considered to be substantially equivalent components for the purposes of illustration, and redundant discussion of those components is omitted for clarity.

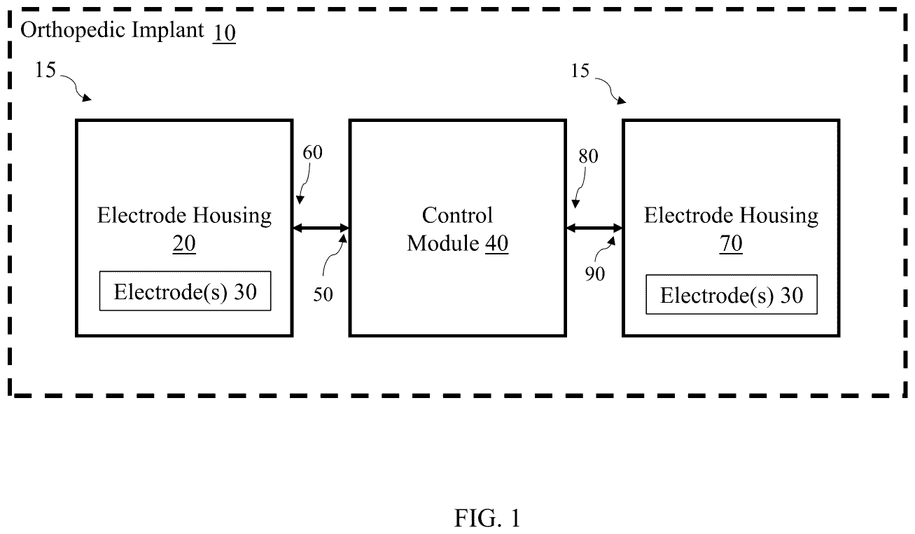

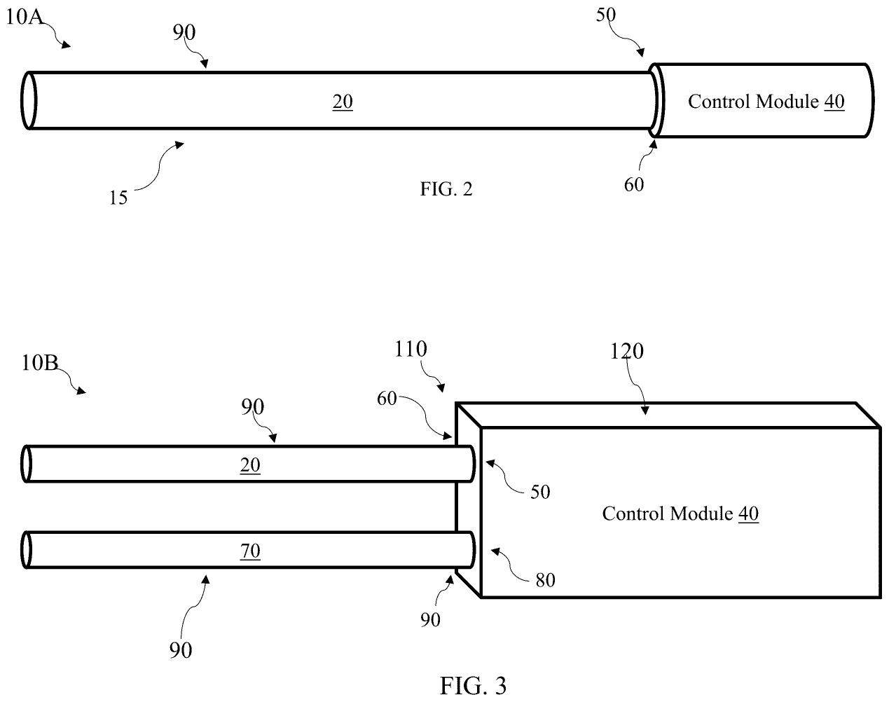

FIG. 1 shows a schematic depiction of an orthopedic implant 10 according to various implementations. Variations on the implant 10 are illustrated in FIGS. 2-6 as implants 10A-10G. The implants 10 are referred to collectively, and can include an electrode assembly 15 that has a first housing 20 including at least one electrode 30, and a control module 40 with a first connector 50 for mating with a complementary connector 60 on the first housing 20. In various implementations, the first housing 20 and the control module 40 include a biocompatible material. In some examples, the biocompatible material includes an electrically insulative biocompatible material. In particular examples, the biocompatible material includes a rigid material such as PEEK (polyether ether ketone), HXLPE (highly cross-linked polyethylene) and/or a flexible material such as silicone, TPU (thermoplastic polyurethane), or PTFE (polytetrafluoroethylene). In additional cases, the biocompatible material includes an implantable metal such as stainless steel or titanium, that is electrically isolated from the electrode(s) 30. As described further herein, the control module 40 can include a controller that is programmed to initiate transmission of a measurement pattern and/or a stimulation pattern to a patient with the electrode(s) 30. In various implementations, the control module 40 includes a hermetically sealed housing constructed from an implantable metal or a biocompatible polymer. In some cases, the implantable metal includes titanium. In certain examples, the biocompatible polymer includes PEEK.

In certain cases, the first housing 20 includes a plurality of electrodes 30. In additional implementations, the implant includes a second housing 70 containing at least one electrode 30, and the control module 40 includes a second connector 80 for mating with a complementary connector 90 on the second housing 70. Connectors can include electrical connectors for enabling electrical connection between electrodes 30 and the control module 40, as well as physical connectors such as clasps, screws, pins, snap-to-fit connectors, press-to-fit connectors, etc. In certain cases, electrical connectors are integrated into physical connectors. FIG. 1 shows an example implant 10 including a control module 40 that is coupled with two distinct housings 20, 70, each including at least one electrode 30 for executing a measurement pattern and/or stimulation pattern, which may including transmitting the pattern to a patient. FIG. 2 shows an example implant 10A with a control module 40 coupled with a first housing 20 that includes at least one electrode (not shown) for executing a measurement pattern and/or stimulation pattern to a patient. Implant 10A can include electrode(s) 30 in a housing 20 that has an arcuate outer dimension 90, e.g., an arcuate outer surface such as a tubular shape having a substantially round or substantially oblong cross sectional geometry. Similarly, the control module 40 can have an arcuate outer dimension 90, e.g., a tubular shape. In certain cases, the control module 40 has a greater outer dimension 90 (or, outer diameter) than that of the electrode housing 20.

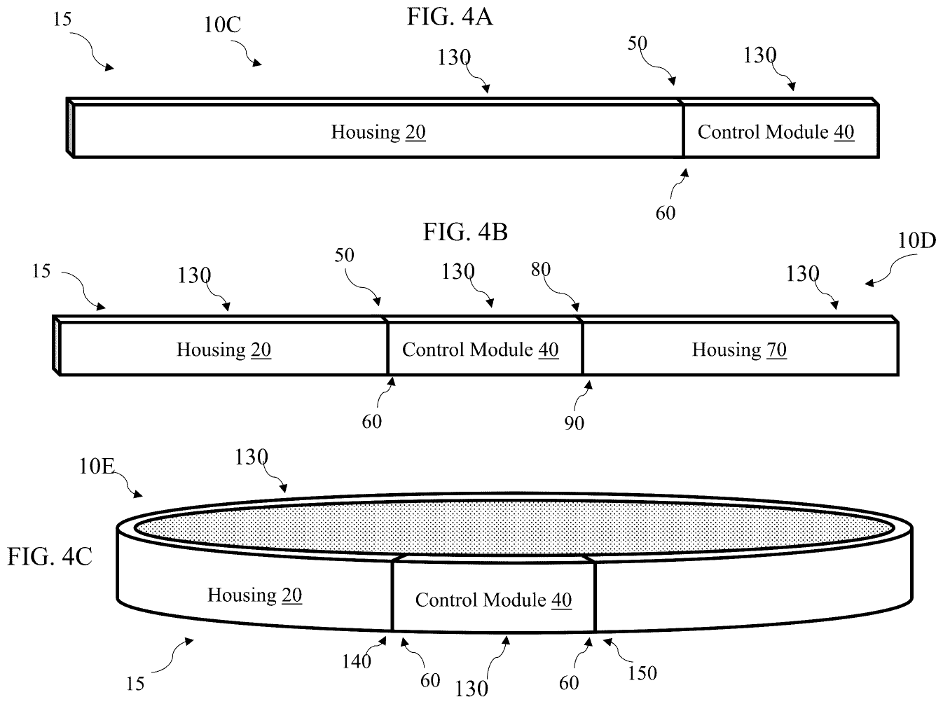

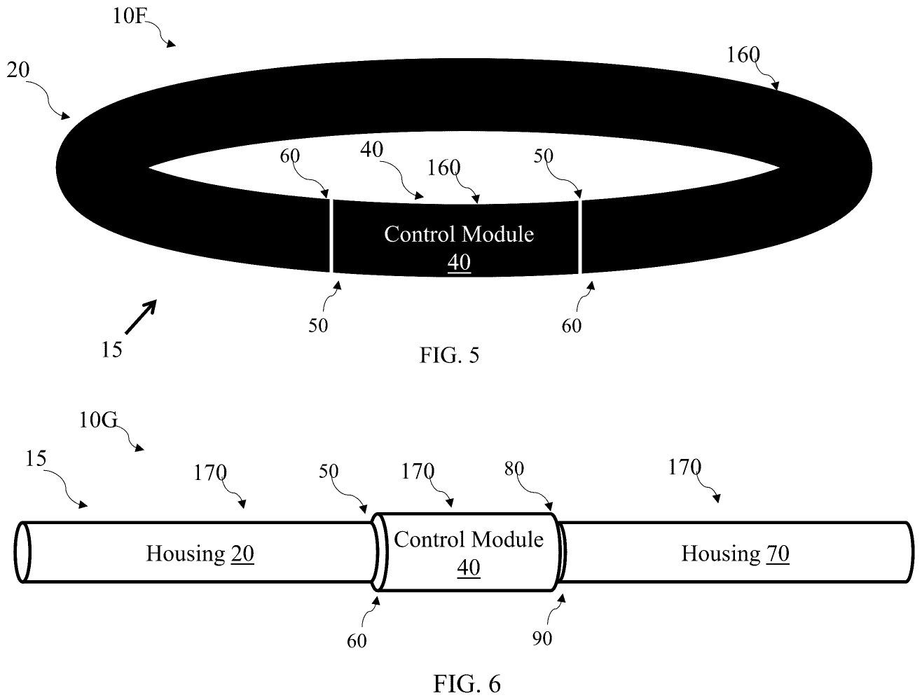

FIG. 3 shows an implant 10B with a control module 40 coupled with two distinct housings 20, 70 on a common side 110 of the control module 40, also for executing a measurement pattern and/or stimulation pattern to a patient. Implant 10B can include electrode housings 20, 70 that have an arcuate outer dimension 90 and a control module 40 having a polyhedral or substantially polyhedral geometry with an outer dimension 120, e.g., an approximately cube, rectangular prism, or three dimensional rounded rectangular prism shape. In certain of these cases, the outer dimension 120 of the control module 40 is greater than the outer dimension 90 (e.g., outer diameter) of each housing 20, 70, and in further cases, is greater than the collective outer dimension of both housings 20, 70. FIGS. 4A-4C show distinct configurations of implants 10C, 10D, and 10E, respectively, with both housing(s) 20, 70 and control modules 40 with angled outer dimensions 130, e.g., squared or rectangular outer dimensions. In certain of these cases, at least one dimension of the housing 20, 70 and control module 40 is greater than another dimension, e.g., a length is greater than a width and/or a depth, or a length is greater than both a width and a depth, or a length is greater than a width, which are both greater than a depth. In certain of these cases, the housing 20, 70 is connected to the control module 40 in a linear arrangement such as, e.g., FIGS. 4A and 4B. In particular examples, such as FIG. 4B, the control module 40 is interposed between electrode assembly housings 20, 70. In other examples, such as FIG. 4C, the control module 40 is connected to the electrode assembly housing 20 at two locations 140, 150, forming a ring shape. FIG. 5 shows another variation on an implant 10F in a ring shape with a central control module 40 and an electrode assembly housing 20 that connects to the housing 20 at two locations 140, 150. In the example implant 10F, the outer dimension 160 of housing 20 and control module 40 is arcuate, e.g., rounded or otherwise non-angular. FIG. 6 shows implant 10G that is similar in linear arrangement to implant 10D (FIG. 4B), but has an arcuate outer dimension 170 in both the housings 20, 70 and control module 40, e.g., the outer dimension 170 is rounded or otherwise non-angular. Various additional form factors of implant 10 are illustrated herein. It is understood that distinct form factors can be combined to provide an implant according to the various implementations herein. Further, while electrode assembly housing(s) 20, 70 are described herein, it is understood that a one or a plurality of electrodes can be independently selected for containment in each housing. For example, a first housing 20 can contain a plurality of electrodes for performing functions described herein. In various implementations, the housing(s) 20, 70 include connectors such as conductive connectors for coupling with the control module 40 and enabling control functions described herein.

In any case, once connected, the first housing 20 (and in some cases, second housing 70 and/or additional housings) and control module 40 of the electrode assembly 15 are sized to complement an orthopedic implant 10. That is, the orthopedic implant 10 can integrate, seat, adhere to, or otherwise retain the electrode assembly 15 to enable implanting and control of the application and/or transmission of measurement and/or stimulation pattern to the patient.

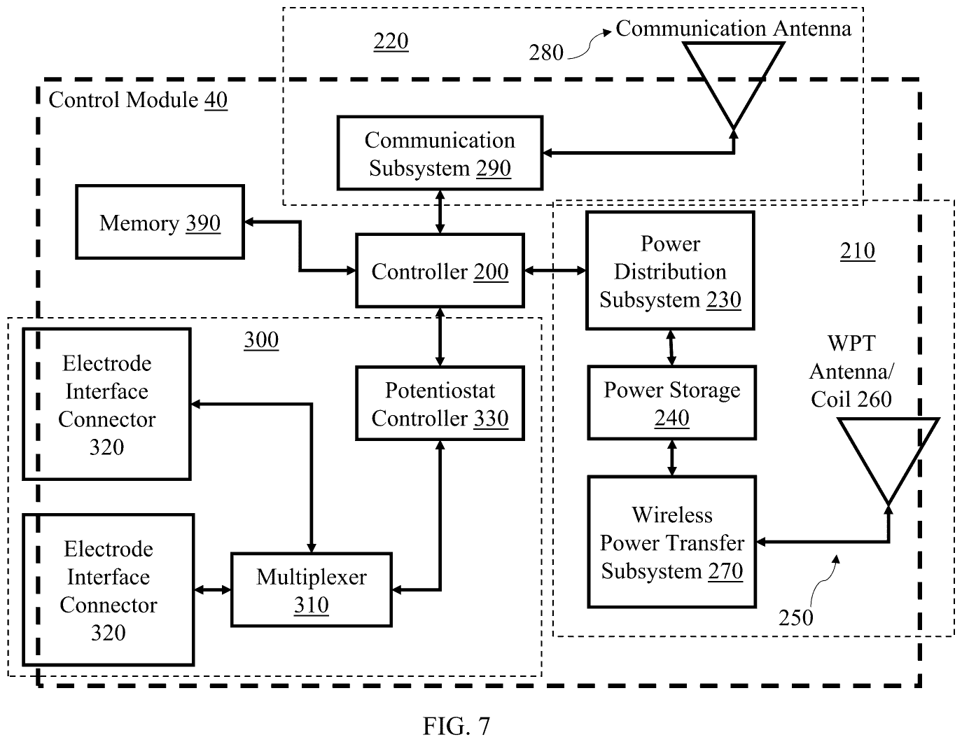

FIG. 7 shows a schematic illustration of example electronics in a control module 40 according to various implementations. In various implementations, the control module 40 includes a controller 200, a power system 210, and a communication system 220. In some examples, the controller 200 includes one or more microcontrollers or microcontroller units (MCU), which in certain cases can include a programmable processor configured (e.g., programmed) to perform functions described herein. In some examples, the controller 200 can include one or more integrated circuits that include one or more of a processor, memory module, communication interfaces, and programmable inputs and outputs. In certain cases, the power system 210 includes a power distribution subsystem 230, power storage 240 (e.g., a battery or capacitor device), and a wireless power transfer (WPT) system 250 including, e.g., a WPT receiver/transmitter 260 such as a WPT antenna or coil, and a WPT subsystem 270. In various implementations, the power system 210 enables WPT to the control module 40 from an external device such as an external power source, e.g., a charging device, a radio frequency (RF) device, etc. The communication system 220 can include a communication antenna 280 and a communication subsystem 290 for managing communication signals from the antenna 280. The communication system 220 enables wireless communication with an external control device, e.g., a smart device such as a smart phone, tablet, computing device, etc. In some cases, wireless communication includes communication with one or more protocols, for example, Bluetooth (BT), BT Low Energy (BLE), radio frequency (RF), etc. The control module 40 can also include an electrode control system 300 that includes a multiplexer 310 for interfacing with electrode interface connectors 320, and in certain cases, a potentiostat controller 330 for controlling execution of the multi-electrode stimulation and/or measurement patterns from the controller 200. As shown, the control module 40 can also include memory 390 (e.g. electrically erasable programmable read-only memory (EEPROM)) for storing instructions such as orthopedic implant measurement and/or stimulation patterns, e.g., instructions for executing a measurement pattern and/or a stimulation pattern via the potentiostat controller 330 and the multiplexer 310. In various implementations, the controller 200 is programmed to initiate transmission of a measurement pattern and/or a stimulation pattern to a patient with at least one of the electrodes 30 (FIGS. 1-6) via the electrode interface connectors 320.

In certain examples, the multiplexer 310 enables individual electrodes to be configured with one or more roles such as the anode or cathode for electrical stimulation functions, or as a working electrode, reference electrode, or counter electrode for electrochemical sensing functions. The multiplexer 310 interfaces with the potentiostat controller 330 which handles the digital-to-analogy conversion of stimulation signals and analog-to-digital conversion of sensor response signals. The potentiostat controller 330 enables the control module 40 to perform a variety of electrochemical measurements using voltammetric techniques such as linear sweep voltammetry, cyclic voltammetry, square wave voltammetry, differential pulse voltammetry, and/or normal pulse voltammetry as well as time-based techniques such as chronoamperometry, pulsed amperometry, open circuit potentiometry, and/or electrochemical impedance spectroscopy. The potentiostat controller 330 can also control the stimulation output in various profiles such as current-controlled, voltage-controlled, monophasic pulsed direct current (DC), biphasic pulsed DC, and alternating current (AC).

In particular cases, the control module 40 is connected with a local control and/or display device such as a smart phone or smart device (e.g., tablet, computing device, etc.) that has network connectivity. In some examples, the local control and/or display device can be configured to communicate over a network to transmit treatment instructions to the control module 40 and/or receive sensor data for remote transmission, e.g., via a network and/or via a cloud-based data connection.

According to various implementations, the controller 200 is configured (e.g., programmed) to initiate transmission of one or both of a measurement pattern or a stimulation pattern to a patient. In certain cases, the controller 200 is configured (e.g., programmed) to initiate transmission of both a measurement pattern and a stimulation pattern to a patient. As described further herein, a given electrode 30 can be configured for use in both the measurement pattern and the stimulation pattern.

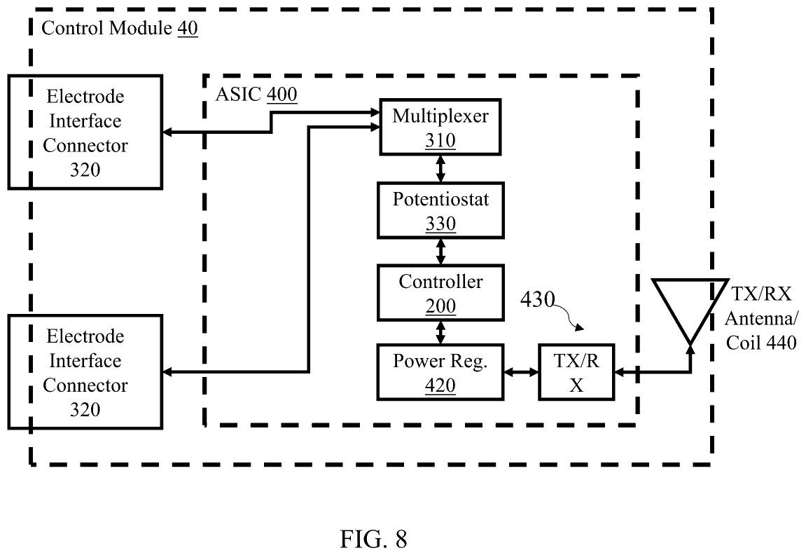

FIG. 8 shows a variation on the control module 40 whereby certain components are integrated in one or more application specific integrated circuits (ASICs) 400. For example, an ASIC can house one or more components in the controller 200, power system 210, and/or communication system 220. In certain cases, features such as the multiplexer 310, potentiostat controller 330, and/or power regulator 420 are housed on the ASIC 400. Further, an interface 430 for a transmitter/receiver antenna/coil 440 can be housed on the ASIC 400 in certain implementations. The use of ASIC(s) 400 can enable miniaturization of certain components in the control module 40, which can enhance adaptability in terms of fit and/or coupling with the body of the implant 10.

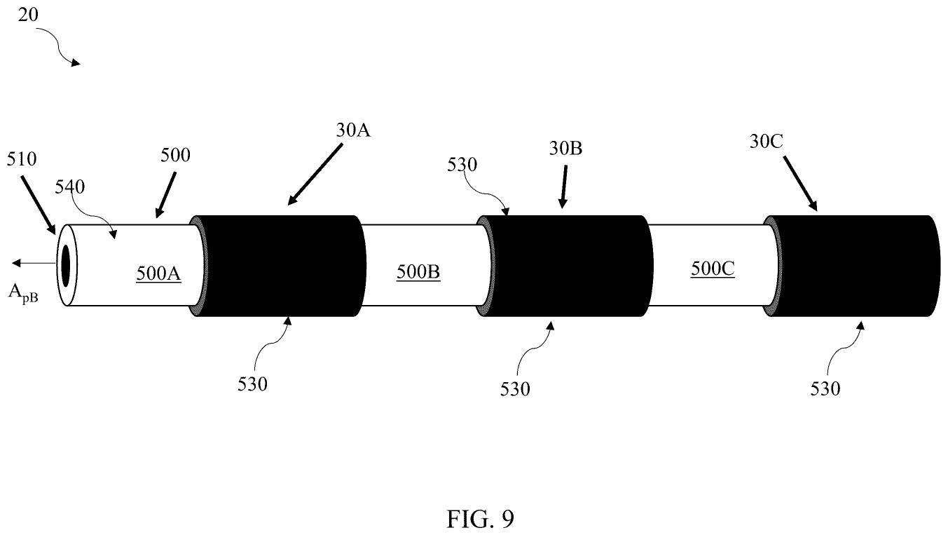

FIG. 9 shows an example schematic depiction of a housing 20 including at least one electrode 30 according to various implementations. In this implementation, the housing 20 includes a body 500 with one or more integrated conductive elements (i.e., electrodes 30) that are electrically isolated from one another. In certain cases, the body 500 includes a channel 510 such as an internal channel that extends along the primary axis (A_{pB}) of the body 500. In certain cases, the channel 510 houses one or more wires and/or conductive traces that enable connection of each electrode 30 with the control module 40. In certain cases, the body 500 includes a single continuous element with terminations at each end, or in other cases, is a toroidal shape (e.g., a ring or loop). In various implementations, multiple electrodes 30 are positioned along the body 500, e.g., two, three, four, five, or more electrodes 30. In certain cases, the body 500 includes an electrically insulative biocompatible material that is rigid (e.g. PEEK, HXLPE, etc.) or flexible (e.g. silicone, TPU, PTFE, etc.). Alternatively, the body 500 may be constructed from an implantable metal (e.g. stainless steel, titanium, etc.) that is electrically isolated from the electrodes 30. The electrodes 30 may be constructed from a noncorrosive conductive implantable metal (e.g. titanium, nitinol, platinum-iridium alloy) or biocompatible carbon-based material (e.g. carbon, graphite, graphene). The exposed surface of the electrode 30 may also include a structural base material (e.g. stainless steel, titanium) coated with a noble metal (e.g. gold, platinum, silver) or carbon-based material (e.g. carbon, graphite, graphene). Additionally, one or more of the electrodes 30 may be functionalized by adding an enzyme layer to couple antibodies, DNA, or proteins that can bind with the target analyte (e.g. bacterial cell) and produce an electrochemical response that can be sensed. In certain cases, as discussed herein, the body 500 can be pliable or flexible to complement distinct shapes and/or slots/grooves in an implant 10. In various implementations, the body 500 is insulative, and includes distinct insulative sections 500A, 500B, 500C, etc., that are interposed between adjacent electrodes 30A, 30B, 30C, etc. In certain cases, electrodes 30 protrude from or extend beyond an outer dimension of the body 500 in at least one direction. For example, as shown in FIG. 9, the outer surface 530 of the electrode 30 extends radially beyond an outer surface 540 of the body 500 (relative to primary axis (A_{pB})). In certain cases, the body 500 is compatible with arcuate embodiments of the electrode assembly, e.g., as shown in FIGS. 2, 3, 5, and 6.

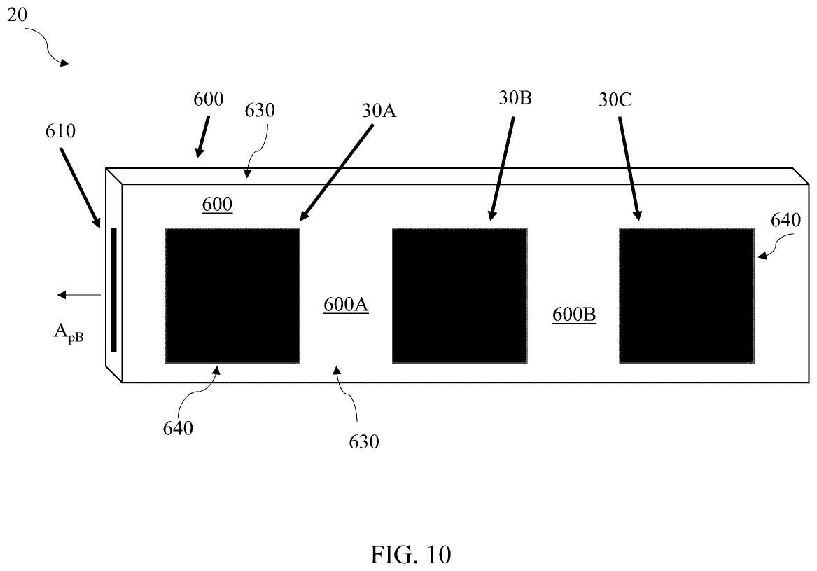

FIG. 10 shows another implementation of a housing 20 including electrodes 30 that are retained in a body 600 that has one or more angular outer surfaces 630, e.g., one or more edges or sides. In certain cases, a plurality of electrodes 30 are arranged linearly along the body 600. The body 600 includes channel 610, such as an internal channel extending along the primary axis (A_{pB}) of the body 600. In certain cases, the channel 610 houses one or more wires and/or conductive traces that enable connection of each electrode 30 with the control module 40. In various implementations, the body 600 is formed of a flexible or pliable material that enables the housing 20 to be bent, twisted, or otherwise manipulated to fit a slot/groove/opening in an implant 10 and/or to comply with a curvature in a surface of an implant 10. In the example implementation of body 600, the electrodes 30 may be evenly or unevenly spaced along the length of the body 600. In certain cases, electrodes 30 coupled with body 600 are rectangular or square electrodes that are evenly spaced along the length of the body 600 (e.g., along primary axis (A_{pB})). In other cases, the electrodes 30 may be rectangular, circular, or polygonal shaped. In various implementations, the body 600 is insulative, and includes distinct insulative sections 600A, 600B, 600C, etc., that are interposed between adjacent electrodes 30A, 30B, 30C. In other cases, the body 600 is formed of a single component with openings for adjacent electrodes 30A, 30B, 30C. In particular cases, outer surfaces 630 of the body 600 are approximately flush with outer surfaces 640 of the electrodes 30, or the outer surfaces 640 of electrodes 30 are recessed relative to the outer surfaces 630 of the body 600. In these examples, the outer surfaces 640 of the electrodes 30 do not extend beyond the outer surfaces 630 of the body 600. In certain cases, the body 600 is compatible with angular embodiments of the electrode assembly, e.g., as shown in FIGS. 4A-4C.

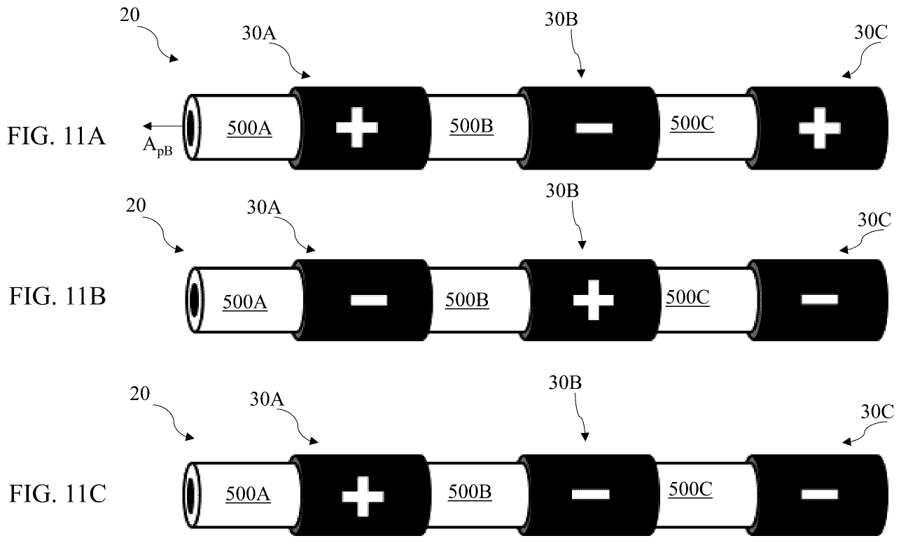

In various implementations, the electrodes 30 (e.g., FIGS. 1-6, 9, 10) are selectable for measurement based on a measurement pattern and/or stimulation based on a stimulation pattern. FIGS. 11-11C illustrate an implementation of a housing 20 including a plurality of electrodes 30 (e.g., three electrodes) separated (or, insulated) from one another by sections 500A of the body 500, with distinct electrode assignments based on a stimulation pattern. With reference to FIG. 11A, a first assignment includes assigning positive polarity to first and third electrodes 30A, 30C and negative polarity to second electrode 30B that is positioned between the first and third electrodes 30A, 30C. A second assignment (FIG. 11B) includes assigning negative polarity to first and third electrodes 30A, 30C and positive polarity to second electrode 30B. A third assignment (FIG. 11C) includes assigning positive polarity to first electrode 30A and negative polarity to second and third electrodes 30B, 30C. In various implementations, positive electrodes act as anodes and negative electrodes act as cathodes. It is understood that during use, the electrode 30 transmits signals from the anode(s), through a material of interest (or, material under test), which can be received (or, detected) at the cathode(s).

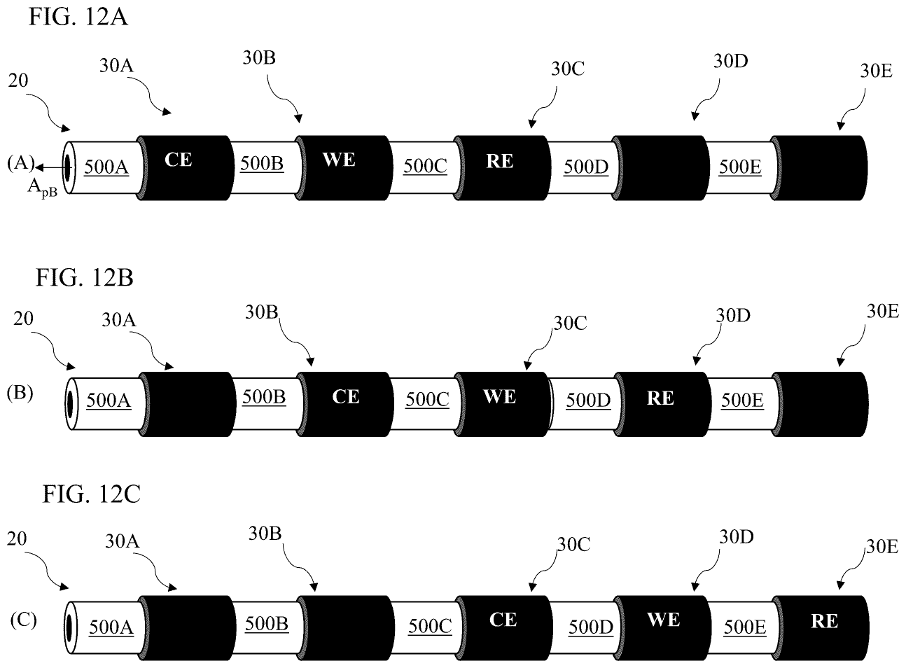

FIGS. 12A-12C show another implementation of a housing 20 including a plurality of electrodes 30 (e.g., five electrodes) separated (or, insulated) from one another by sections 500A-E of the body 500, with distinct electrode assignments based on an applied measurement pattern. It is understood that this housing 20 can be used as part of an electrochemical cell for conducting measurement on a patient. Further, it is understood that this housing 20 can be used for both stimulation as described with respect to FIGS. 11A-11C, as well as for measurement, e.g., with the controller 200 dictating the polarity and/or assignment of electrodes 30 for use in one or more patterns.

In the example of FIGS. 12A-12C, each electrode 30 can be directed as the working electrode (WE), a counter electrode (CE), or reference electrode (RE) to form an electrochemical cell and enable sensor measurements at each electrode location in the assembly. An example sequential chain of changing electrode role is presented in the progression from FIG. 12A, to FIG. 12B, to FIG. 12C, whereby the role of electrodes 30A-E change according to a prescribed measurement pattern. In this example, in progression from FIG. 12A to FIG. 12C, the working electrode is switched from electrode 30B to electrode 30C, and then to electrode 30D, while counter electrodes and reference electrodes are switched from 30A to 30B to 30C and 30C to 30D to 30E, respectively. In various implementations, when performing measurement (e.g., electrochemical sensing) functions, each electrode 30 of the assembly may be used as the working electrode (i.e. sensing electrode), while the adjacent electrodes act as the counter electrode (CE) and reference (or pseudo-reference) electrode (RE) to complete the electrochemical cell. In particular examples, measurements can be captured in series at every electrode 30 by alternating which electrodes 30A, 30B, 30C, etc. are used as the working, counter, and reference electrodes. In a similar manner, as described with reference to housing 20 in FIGS. 9-11, each electrode 30 may serve as the anode or cathode in a two-electrode stimulation treatment system, e.g., to apply a stimulation pattern. By adjusting the stimulation program and directing which electrodes 30 are the anode and cathode, the user/operator can design a stimulation protocol to target a treatment based on clinical need.

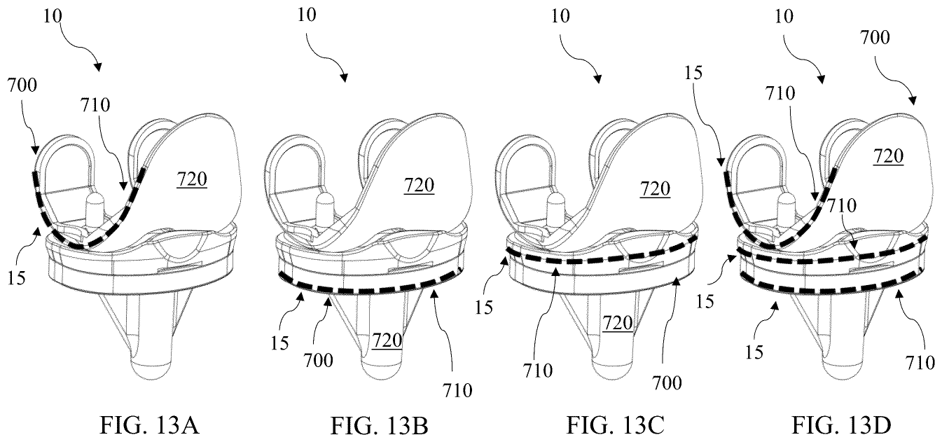

FIGS. 13A-13D illustrate various depictions of an implant 10 including integrated electrode assemblies 15 according to various implementations. In this implementation, the implant 10 includes a knee implant such as a total knee implant. It is understood that the electrode assemblies 15 illustrated and described according to various implementations can also be applied to various other implants. For example, in addition to knee implants, the assemblies 15 can be particularly suited for integration with at least one of a tibial insert, a hip implant, or a surgical nail.

In these examples, implant 10 can have at least four distinct integration features, shown in FIGS. 13A through 13D for mounting, adhering, or otherwise affixing the electrode assemblies 15 illustrated herein. Various other integration features are also possible. In the examples shown, one or more cylindrical electrode assembles 15 may be arranged in or on the surface 700 of an orthopedic implant 10. For example, the cylindrical assembly 15 can fit within a groove or slot 710 in the implant body 720. In certain cases, the groove or slot 710 includes a semicircular groove or slot, and/or includes a lip for retaining the assembly 15. In particular examples, the assembly 15 is sized to enable a snap-to-fit or press-to-fit connection with the body 720 at the groove or slot 710. In certain examples, the groove or slot 710 is recessed from the outermost surface of the body 720, such that a portion of the assembly 15 (e.g., an electrode) does not protrude from the outermost surface of the body 720. In particular examples, the assembly 15 sits approximately flush with the outermost surface of the body 720 to enable contact with the patient's tissue once implanted. In some cases, each assembly 15 may be preassembled with the implant components as part of the manufacturing assembly process. Alternatively, each electrode assembly 15 may be manufactured apart from the implant components and assembled by the surgical team in the operating theater. In particular cases, the assembly 15 includes at least one flexible insulative section (e.g., such as sections 500A, 500B, etc., FIG. 9) that enables the assembly 15 to complement contours in the implant body 720. In certain cases, the flexible insulative sections enable an operator (e.g., medical professional) to adjust the contour of the assembly 15 to the implant body 720 during or prior to a surgical procedure. In further examples, components of an electrode assembly 15 (including housing 20 and control module 40) are provided separately and assembled during the surgical procedure, e.g., in the surgical theater or intraoperatively. In certain examples, as noted herein, each housing 20 has an electrical connector component with one or more conductive surfaces (e.g., pads) that are individually electrically coupled to each electrode 30. In certain cases, the housing 20 is securely attached to mating contacts on the control module 40, e.g., on the housing or canister of the control module 40 via mechanical means such as with set screws or pins, connections of which can be made in the surgical theater or intraoperatively.

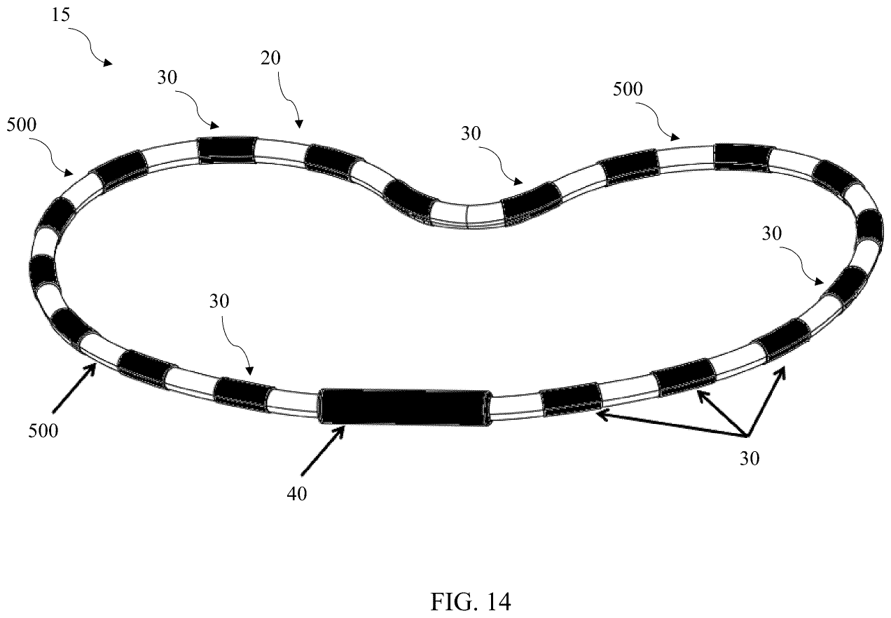

FIG. 14 shows a detailed view of an electrode assembly 15 such as is shown in FIG. 13D, including a flexible housing 20 (including body sections 500 and electrodes 30) coupled with a control module 40 at two sides to form a flexible band. A flexible band implementation can be beneficial because it is compliant to match the curvature of the tibial insert geometry, can snap into a mating feature (e.g. groove or slot) on the tibial insert, and can stretch to fit a variety of tibial insert sizes and types. In various implementations, the flexible band enables both adjustment of shape and size, e.g., it can be shaped to fit the geometry of an implant and also expand (or flex) to fit around portions of an implant. In certain cases, the housing 20 has elastic properties enabling a stretch-to-fit application of the assembly 15. In various implementations, the flexible band assembly, e.g., assembly 15 in FIG. 14 can attach to a tibial insert via a mating feature machined into the outer surface of the implant body. In certain cases, the mating feature is machined to complement a size of the assembly 15, e.g., an outer dimension of the assembly 15.

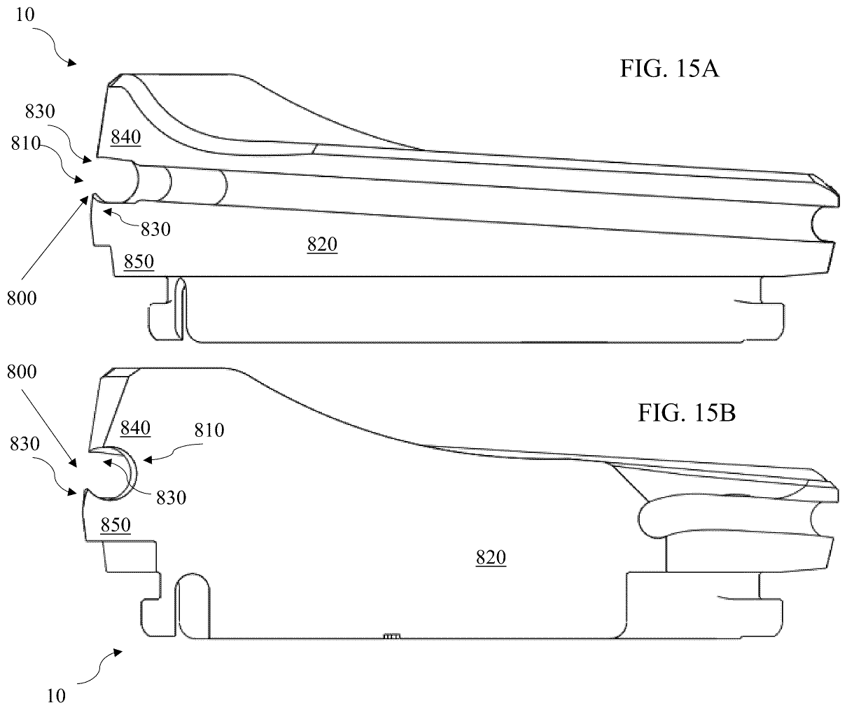

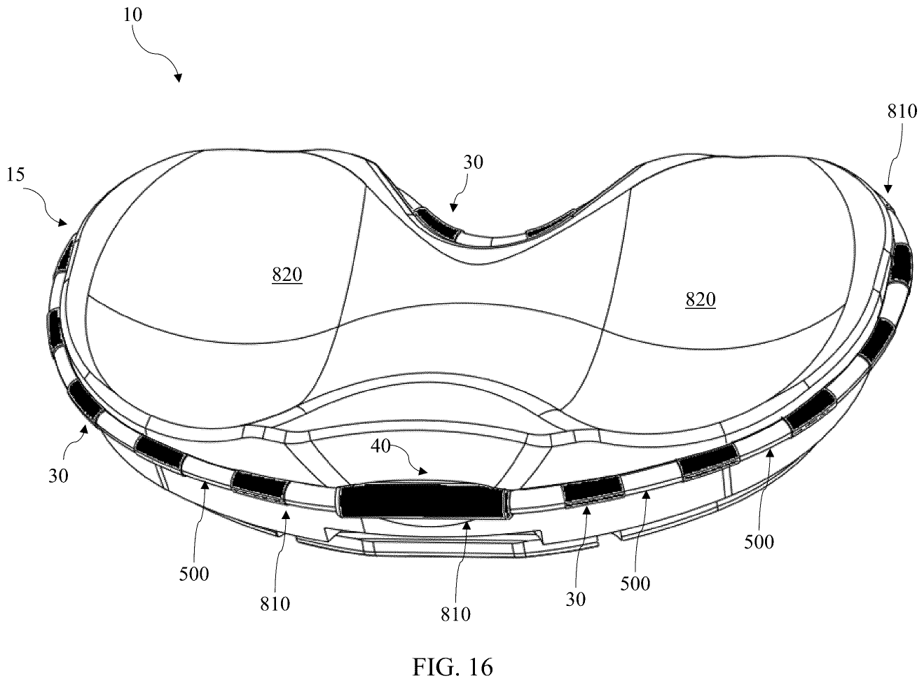

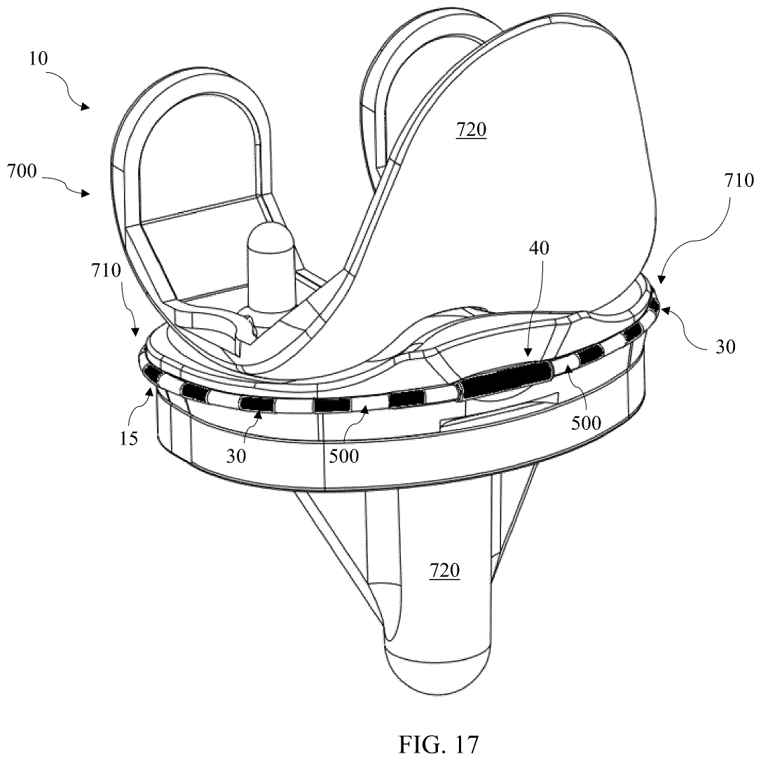

A particular depiction of a mating feature 800 in an implant 10 is illustrated in FIGS. 15A and 15B, which show a tibial implant in side and cross-sectional views, respectively. The mating feature 800 in this example includes a semi-circular groove or slot 810 in the body 820. A semi-circular groove or slot can be beneficial for creating a cavity to house and retain the assembly 15. A retention feature 830 such as a protruding edge on the upper portion 840 and/or lower portion 850 of the groove or slot 810 enables retention of the assembly 15, e.g., in a snap-to-fit or press-to-fit application. During assembly, the assembly 15 can be deformed (without damage) to fit between the retention feature 830 to snap into the groove or slot 810. The retention features 830 prevent unwanted dislodging of the assembly 15 during use. In some cases, removal of the assembly 15 from the groove or slot 810 can only be performed with a tool. FIG. 16 illustrates a flexible band-type assembly 15 (FIG. 14) assembled with the tibial insert implant 10 (FIGS. 15A-15B) according to various implementations. FIG. 17 shows the flexible band-type assembly 15 (FIG. 15) assembled with the knee implant of FIG. 13C.

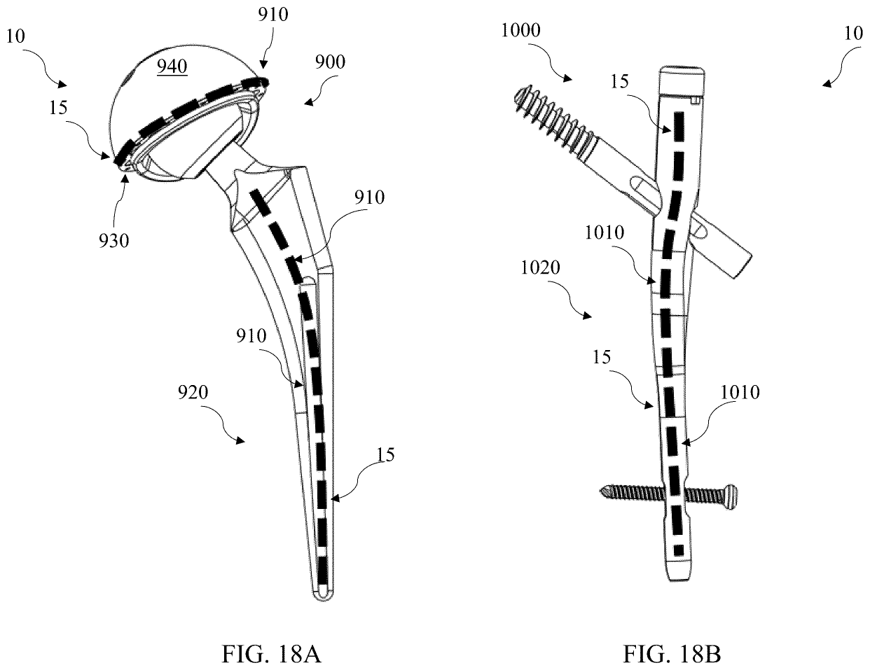

Certain applications, such as on the rim of the femoral component shown in FIG. 18A, may benefit from a cylindrical electrode body 30 with an integrated control module 40 similar to configurations of an electrode assembly 15 shown in FIGS. 2 and 3. Other applications of such an assembly 15 configuration include hip arthroplasty and trauma nailing. In a total hip replacement application such as illustrated in the hip implant 900 shown in FIG. 18A, the mating feature (e.g., slot or groove) 910 can be located along the length of the femoral stem 920 to accept an electrode assembly 15 such as a cylindrical electrode body, or the mating feature 910 can be located along circumferential edge 930 of the acetabular cup 940 to accept a flexible band electrode assembly 15. In a trauma nailing application, such as a troch nail 1000 shown in FIG. 18B, a mating feature 1010 can be located along the length of the nail shaft 1020 to accept a cylindrical electrode assembly 15 with integrated control module 40.

In any of the previously described applications and arrangements, it may be preferable for the form of the electrode assembly 15 (including integrated control module 40) to be of a non-circular cross-section, e.g., as illustrated in FIG. 4. As noted herein, one alternate embodiment may be a rectangular cross-section.

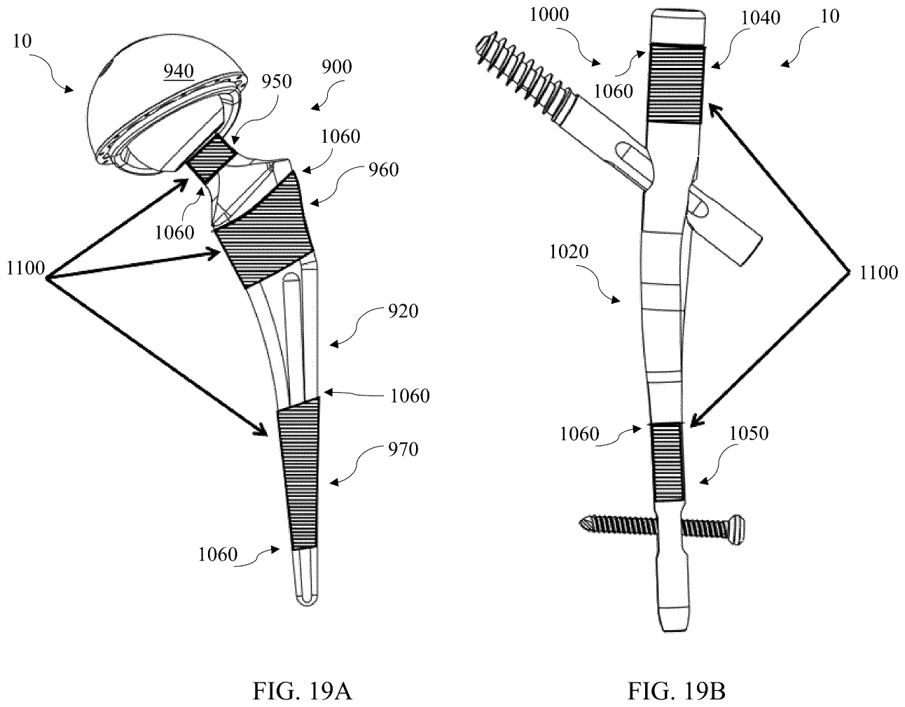

In certain other applications, it may be preferable for one or more of the previously described electrode assemblies to be integrated in a flexible tubular housing. A tubular embodiment can enable a flexible tubular assembly to be attached to a shaft-like implant component without the need for mating features to be machined for each electrode assembly. In a total hip replacement application, e.g., as shown in FIG. 19A, tubular electrode assemblies 1100 may be attached around one or more of the neck 950, superior portion 960, or inferior portion 970 of the femoral stem 920. In a trauma nailing application (FIG. 19B), such as a troch nail 1000, the tubular electrode assemblies 1100 may attach around the superior 1040 or inferior 1050 portions of the nail shaft 1020. In both applications, a feature on the implant body may be beneficial to prevent axial migration of tubular electrode assembly 1100 with respect to the implant component during and after implantation. One such feature may consist of a recess or trench 1060 in which the flexible tubular electrode assembly 1100 can sit. In certain cases, the recess or trench 1060 is wider than a width of the electrode assembly 1100, which can enable the assembly 1100 to be wrapped around the body and retained by its own elasticity within the recess or trench 1060. In certain examples, the recess or trench 1060 is defined by at least two edges or sides that can axially retain the assembly 1100.

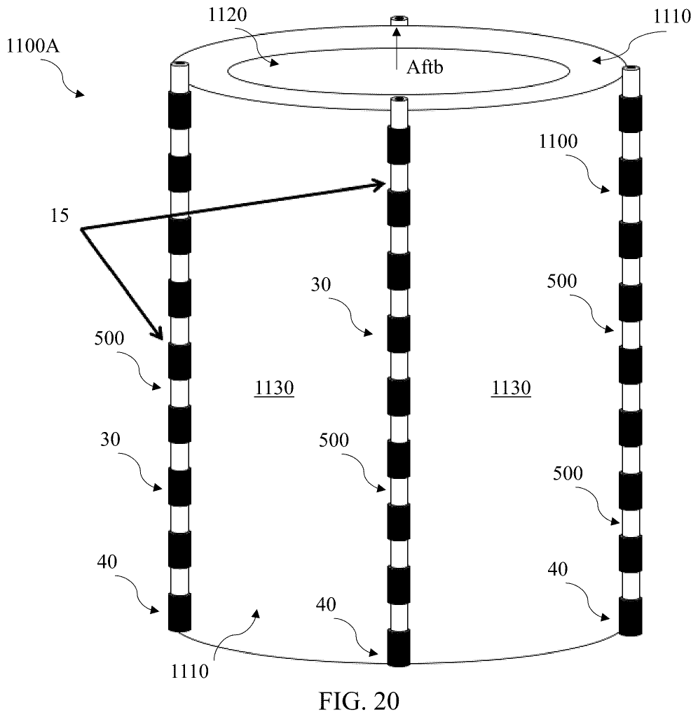

An example of a first type of flexible tubular assembly 1100A is illustrated in FIG. 20, including a flexible body 1110, and a plurality of cylindrical electrode assemblies 15 similar to electrode assemblies described herein. The electrode assemblies 15 can each include a set of body members 500 interposed between electrodes 30, and in certain cases, each coupled with a control module 40. In other cases, a central or shared control module 40 can be positioned within the body 1110 (e.g., in a channel 1120) or at a terminal end of the body 1110 and coupled with one or more electrode assemblies 15. In certain cases, a plurality of cylindrical electrode assemblies 15 are coupled with a wall 1130 (or walls) of the body 1110. In certain examples, at least one electrode 30 is integrated in the wall 1130 of the flexible tubular body 1110 such that a primary axis of each electrode is approximately parallel with a primary axis (A_{ftb}) of the flexible tubular body 1110.

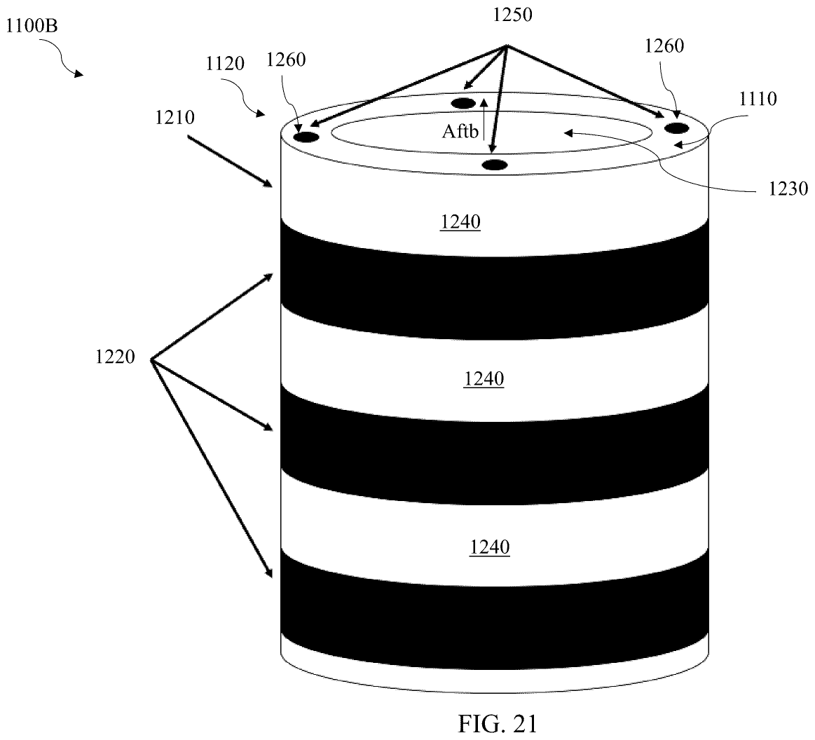

In another implementation, as shown in FIG. 21, a flexible tubular assembly 1100B includes a flexible body 1210 and a plurality of annular electrodes 1220 arranged around the flexible body 1210. In these implementations, the body 1210 includes an internal channel 1230, and at least one of the electrodes 1220 is integrated in a wall 1240 of the body 1210 such that a primary axis of the electrode 1220 is off-axis relative to a primary axis (A_{ftb}) of the body 1210, e.g., approximately perpendicular to at least 5 degrees off-axis. The example in FIG. 21 shows electrodes 1220 running perpendicular to the primary axis (A_{ftb}) of the body 1210. In certain cases, adjacent electrodes 1220 are evenly separated (spaced) relative to one another, and in other cases, are unevenly separated (spaced). In particular examples, the electrodes 1220 are integrated directly within the walls 1240 of a flexible tubular body 1210, e.g., in slots or gaps. In addition, the channel 1230 can be configured to house (and in some cases, insulate) wires or contacts between each electrode and an integrated control module. In certain of these cases, a single control module (not shown) is housed in the channel 1230 to control the electrodes 1220. In a particular example, wire channels 1250 are integrated in the walls 1240 (e.g., extending axially along (A_{ftb}) enabling routing of wires 1260 between electrodes 1220 and the control module(s).

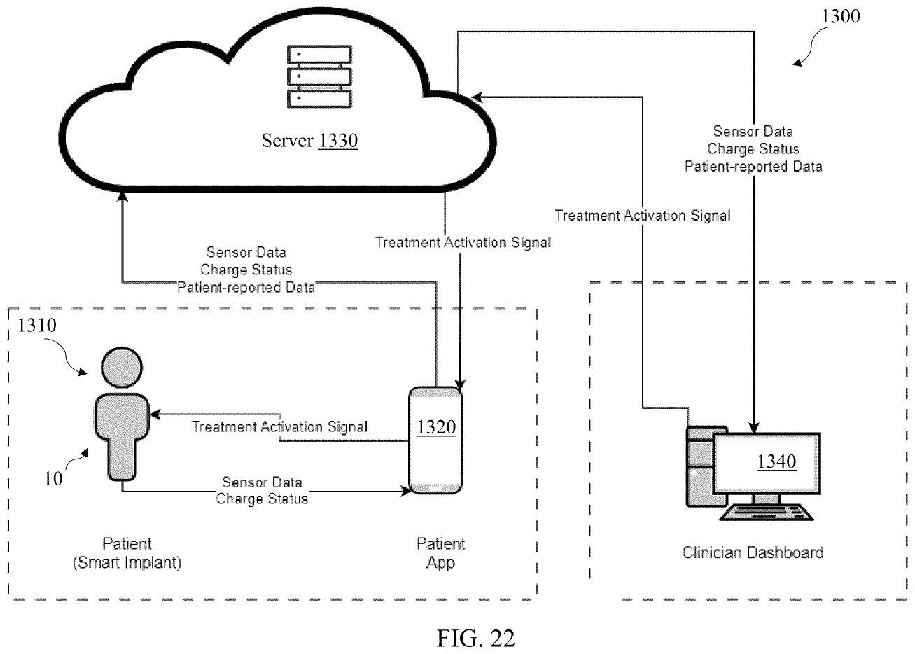

In particular cases, the implants 10 shown and described herein can be part of a “smart” implant system or platform, as illustrated in the system 1300 in FIG. 22. In these implementations, a patient 1310 having an implant 10 such as those described herein can be provided with a diagnostic or interventional treatment such as a measurement and/or sensing treatment via a smart device 1320, which may be connected via a network such as a cloud server 1330 with a clinician dashboard 1340. The dashboard 1340 can include an interface enabling a clinician to send a treatment activation signal through the network 1330 to the smart device 1320 to activate treatment at the implant 10 in the patient 1310. The dashboard 1340 can also be configured to receive data such as sensor data, charge status, and/or patient reported data (via network 1330) from the smart device 1320, which in turn receives sensor data and/or charge status data from the implant 10. In one implementation of a smart implant control system, the implant 10 communicates with the patient's smart device 1320 over a 2.4 GHz protocol such as Bluetooth or Bluetooth Low Energy. A smart device-compatible dongle may also be used to communicate to the implant 10 on a radiofrequency (RF) band that is not directly supported by the smart device 1320, such as 915 MHz or 401-406 MHz (MICS). The smart device dongle can contain transmission and receiver antenna(s) tuned to the target frequency of the implant communication protocol. In various examples, data is sent from patient's implant 10, received by the integrated antenna or dongle's antenna at the smart device 1320, and then processed through a dedicated software application (or, app) on the patient's smart device 1320. The data can then be uploaded from the patient's smart device 1320 to a secure server/network via internet connection such as WiFi or LTE. Data may be processed on the patient's smart device 1320 prior to upload or on the server after upload. After processing, the data can then be accessed by the patient via their software application and by a clinician via a dedicated clinician dashboard 1340. The clinician may choose to activate a treatment protocol and remotely deliver the treatment signal through this wireless connectivity infrastructure.

Devices such as implants described herein are described as including communication devices and related electronics. These communications device(s) can include one or more transmitters and/or receivers (e.g., wireless and/or hard-wired transmitters/receivers). In various implementations, the communication devices are configured for a plurality of communication protocols, e.g., wireless protocols such as Wi-Fi, Bluetooth, BLE, Zigbee, etc., as well as radio communication and intercom communications, and/or a hardwired connection (e.g., fiber optic connection).

As noted herein, the implants and related electrodes disclosed according to various implementations provide numerous benefits relative to conventional implant apparatuses and systems. For example, various implementations can reduce complexity and/or invasiveness of conventional surgical procedures, e.g., by attaching the electrodes on or within the surface of the implant form. Further, relative to conventional implants, the disclosed implementations efficiently allocate electrodes and control modules, and also provide one or more separate components that can be assembled to traditional implant components within the surgical theater. Various implementations include assemblies, implants, systems and methods of coupling electrodes to smart orthopedic implant systems. Relative to conventional implants, the disclosed implants, assemblies and systems can improve patient outcomes and ease monitoring and/or treatment of patients post-operatively.

The functionality described herein, or portions thereof, and its various modifications (hereinafter “the functions”) can be implemented, at least in part, via a computer program product, e.g., a computer program tangibly embodied in an information carrier, such as one or more non-transitory machine-readable media, for execution by, or to control the operation of, one or more data processing apparatus, e.g., a programmable processor, a computer, multiple computers, and/or programmable logic components.

The term “approximately” as used with respect to values herein can allot for a nominal variation from absolute values, e.g., of several percent or less. Where the term “comprising” is used in the present description and claims, it does not exclude other elements or operations. The term “based on” (as in “A is based on B”) is used to indicate any of its ordinary meanings, including the cases (i) “based on at least” (e.g., “A is based on at least B”) and, if appropriate in the particular context, (ii) “equal to” (e.g., “A is equal to B”). Similarly, the term “in response to” is used to indicate any of its ordinary meanings, including “in response to at least.”

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Actions associated with implementing all or part of the functions can be performed by one or more programmable processors executing one or more computer programs to perform the functions of the calibration process. All or part of the functions can be implemented as special purpose logic circuitry, e.g., an FPGA and/or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. Components of a computer include a processor for executing instructions and one or more memory devices for storing instructions and data.

In various implementations, components described as being “coupled” to one another can be joined along one or more interfaces. In some implementations, these interfaces can include junctions between distinct components, and in other cases, these interfaces can include a solidly and/or integrally formed interconnection. That is, in some cases, components that are “coupled” to one another can be simultaneously formed to define a single continuous member. However, in other implementations, these coupled components can be formed as separate members and be subsequently joined through known processes (e.g., soldering, fastening, ultrasonic welding, bonding). In various implementations, electronic components described as being “coupled” can be linked via conventional hard-wired and/or wireless means such that these electronic components can communicate data with one another. Additionally, sub-components within a given component can be considered to be linked via conventional pathways, which may not necessarily be illustrated.

While inventive features described herein have been described in terms of preferred embodiments for achieving the objectives, it will be appreciated by those skilled in the art that variations may be accomplished in view of these teachings without deviating from the spirit or scope of the invention. Also, while this invention has been described according to a preferred use in spinal applications, it will be appreciated that it may be applied to various other uses desiring surgical fixation, for example, the fixation of long bones.

A number of implementations have been described. Nevertheless, it will be understood that additional modifications may be made without departing from the scope of the inventive concepts described herein, and, accordingly, other implementations are within the scope of the following claims.

We claim:

1. An electrode assembly comprising:
a first housing containing at least one electrode; and
a control module with a first connector for mating with a complementary connector on the first housing, the control module including a controller programmed to initiate transmission of at least one of a measurement pattern or a stimulation pattern to a patient with the at least one electrode,
wherein the first housing and the control module include a biocompatible material, and when connected, are sized to complement an orthopedic implant.

2. The electrode assembly of claim 1, wherein the controller is programmed to initiate transmission of both the measurement pattern and the stimulation pattern to the patient, and wherein the at least one electrode is configured for use in both the measurement pattern and the stimulation pattern.

3. The electrode assembly of claim 1, wherein the first housing contains a plurality of electrodes.

4. The electrode assembly of claim 1, further comprising a second housing containing at least one additional electrode, wherein the control module includes a second connector for mating with a complementary connector on the second housing.

5. The electrode assembly of claim 1, wherein when the first housing and the control module are connected, the electrode assembly is pliable to complement at least one contour in the orthopedic implant.

6. The electrode assembly of claim 5, wherein the electrode assembly is sized to sit within a groove or slot in the orthopedic implant.

7. The electrode assembly of claim 6, wherein the first housing includes a flexible insulative section adjacent the at least one electrode.

8. The electrode assembly of claim 1, wherein the first housing includes a plurality of electrodes interposed between adjacent insulative sections, wherein each of the plurality of electrodes is selectable for at least one of: measurement based on the measurement pattern or stimulation based on the stimulation pattern.

9. The electrode assembly of claim 8, wherein the plurality of electrodes includes at least three electrodes,
wherein according to the stimulation pattern: a first electrode is selected as an anode and a second electrode is selected as a cathode, and
wherein according to the measurement pattern: a first electrode is selected as a working electrode, a second electrode is selected as a counter electrode, and a third electrode is selected as a reference electrode.

10. The electrode assembly of claim 9, wherein the controller is configured to change selection of one or more of the plurality of electrodes according to the stimulation pattern or the measurement pattern.

11. The electrode assembly of claim 1, wherein the first housing includes a flexible tubular body and wherein the at least one electrode is integrated in a wall of the flexible tubular body such that a primary axis of each electrode is parallel with a primary axis of the flexible tubular body.

12. The electrode assembly of claim 1, wherein the first housing includes a flexible tubular body with an internal channel, and wherein the at least one electrode is integrated in a wall of the flexible tubular body such that a primary axis of each electrode is off-axis relative to a primary axis of the flexible tubular body.

13. The electrode assembly of claim 1, wherein the control module includes a hermetically sealed housing constructed from an implantable metal or a biocompatible polymer, wherein the housing includes:
the controller, memory, a multiplexer, a power system, and a communication system, wherein the power system enables wireless power transfer to the control module and wherein the communication system enables wireless communication with an external control device.

14. The electrode assembly of claim 1, wherein the first housing and the control module have a substantially arcuate outer dimension or a substantially angled outer dimension.

15. The electrode assembly of claim 1, wherein the orthopedic implant includes a knee implant, a tibial insert, a hip implant, or a surgical nail.

16. The electrode assembly of claim 1, wherein the first housing and the control module are integrally assembled, or configured for assembly during a surgical procedure on the patient.

17. An orthopedic implant comprising:
a body for securing in a patient, the body including a mating feature; and
an electrode assembly coupled with the body at the mating feature, the electrode assembly including:
a first housing containing at least one electrode; and
a control module connected with the first housing, the control module including a controller programmed to initiate transmission of at least one of a measurement pattern or a stimulation pattern to a patient with the at least one electrode,
wherein the electrode assembly complements the mating feature on the body.

18. The orthopedic implant of claim 17, wherein the first housing includes a plurality of electrodes interposed between adjacent insulative sections, wherein each of the plurality of electrodes is selectable for at least one of: measurement based on the measurement pattern or stimulation based on the stimulation pattern.

19. The orthopedic implant of claim 17, wherein the controller is programmed to initiate transmission of both the measurement pattern and the stimulation pattern to a patient, and wherein the at least one electrode is configured for use in both the measurement pattern and the stimulation pattern.

20. The orthopedic implant of claim 17, wherein the mating feature includes a groove or a slot in the body, and wherein the electrode assembly is retained in the groove or slot in the body.
